(19) United States
(12) Patent Application Publication
(10) Pub. No.: US 2010/0030341 A1
(43) Pub. Date: Feb. 4, 2010

(54) SYSTEM LINER AND MYOELECTRONIC ELECTRODE UNIT SYSTEM

(75) Inventors: Hans Dietl, Gablitz (AT); Horst Willburger, Wien (AT); Hermann Lanmuller, Vienna (AT)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: OTTO BOCK HEALTHCARE PRODUCTS GmbH, WIEN (AU)

(21) Appl. No.: 11/911,975

(22) PCT Filed: Apr. 26, 2006
(86) PCT No.: PCT/EP2006/003873

§ 371 Date: Mar. 28, 2008

(30) Foreign Application Priority Data
May 4, 2005 (DE) 10 2005 021 412.6

(51) Int. Cl. A61F 2/70; A61F 2/78

(52) U.S. Cl. 623/25; 623/32

(57) ABSTRACT

The invention relates to a myoelectrical electrode unit for a prosthetic device, which includes a liner positioned between an amputation stump and the prosthetic device and a myoelectrical electrode unit for detecting and transmitting myoelectrical signals to control the prosthetic device.

The invention relates to a system consisting of a liner positioned between an amputation stump and a prosthesis shaft and a myoelectrical electrode unit for the creation of a myoelectrical signal for controlling prosthesis on an amputation stump.

A myoelectrode serves for the acquisition and evaluation of a surface myogram, based on which motor driven elements of a prosthesis are controlled. The overall functionality of such a prosthesis, in particular an arm prosthesis, is therefore immediately dependent on the quality of the myoelectrode. Active myoelectrodes are known in the state of the art of technology which consists of conduction electrodes for the conduction of the electromyogram as well as of signal processing elements.

The myoelectrodes previously produced in a closed plastic housing consist of an entry interface positioned on the amputation stump and an exit interface to the prosthesis. The entry interface is formed on metal myoelectrodes on the surface of the housing. The exit interface makes the strengthened and processed electromyogram signal available to the prosthesis in analog or digital form.

In recent years there has been a development in prosthetic care technology of a so called liner-shaft technology. The patient wears a soft sock like unit over the amputation stump, different from regular prosthesis shafts. This unit is designated as the liner. The patient wears the prosthesis over the liner. The liner forms a bond between the stump and the prosthesis which on one hand improves the position of the prosthesis shaft and on the other hand increases the comfort of the prosthesis. The liner is made of materials such as silicone or polyurethane and has excellent adhesion properties to the stump which is significantly improved by the airtight seal of the liner to the amputation stump.

The currently known, cable attached myoelectrodes are not able to be used with the liner technology. Because these myoelectrodes are hung elastically in the shaft, a window would have to be cut in the liner in order to create the contact between the liner and the stump. This would result in a reduction of the adhesion between the liner and the stump. In addition, the liner would have to be exactly positioned on the stump so that the window in the liner is exactly lined up with the position of the myoelectrode in the prosthesis shaft.

The task of the invention is to create a system with which the known disadvantages in the state of the art technology can be avoided. Accordingly, this is solved by a system with the characteristics in claim 1, including a measurement unit which is positioned on the stump side of the liner and a receptor unit which is on the side of the liner facing away from the stump, whereby the measurement unit has a sender for the wireless signal transmission of the myoelectrical signal to the receptor unit. Thus it is possible, on the one hand, to utilize the advantages of the liner technology relating to wear comfort and adhesion properties and on the other hand allows the use of a prosthesis with myoelectrical controls.

There is a telemetric myoelectrode present that consists of two units, of which one unit within the liner has contact with the amputation stump, and one receptor unit between the liner and the prosthesis shaft, preferably mounted on the prosthesis shaft.

The invention also has an amplifier in the measurement unit for amplification of the myoelectrical signals, an analog digital converter for digitalization of the signal as well as a coding unit for coding of the signal, in order to transmit the amplified, digitized and coded signal wirelessly via transmission unit. The receptor unit conducts the received myoelectrical signal for further processing within the prosthesis. The measurement unit can be attached to the inner side of the liner, for example, glued or welded, in order to enable a problem free positioning of the liner. The measurement unit is preferably integrated directly into the liner as a flat water tight capsule. The telemetric myoelectrical method allows the use of a closed liner within the prosthesis shaft which separates the mechatronic components of the prosthesis from the amputation stump.

In order to create a measuring unit that is free of wear and tear and maintenance free, an induction spool or a unit for conversion of electromagnetic conduction conversion fields is attached so that energy sources such as batteries can be avoided. The energy transmission occurs via an electromagnetic alternating current which is generated in the receiving unit so that a telemetric energy transmission is made.

In addition to transmission of the myoelectrical signal through electromagnetic waves, the sender is optically formed and the receiver in the receiving unit is an optical sensor in order to enable optical signal transfer. In the measurement unit a light diode can be included, which sends the digitized binary signal to the conduction through an in/out modulation of the efficacy of the receiver unit on the liner.

The signal transmission can also occur via frequency modulated load modulation; with this the transmission of the power supply is linked to the transmission of the electromyogram signal. The measurement unit detunes the resonance circuit which is used for transmission of the energy. The detuning is modulated with the binary signal flow. This detuning is detected and demodulated in the reception unit.

The signal transmission can also occur via amplitude modulated load modulation; with this the transmission of the power supply is linked to the transmission of the electromyogram signal. The measurement unit dampens the resonance circuit which is used for transmission of the energy. The dampening is modulated with the binary signal flow. This dampening is detected and demodulated in the reception unit.

It has also been provided that the signal transmission is done via amplitude modulated carriers. In this, a carrier created in the sender is modulated with the binary data flow. The carrier will be sent via its own electromagnetic connection independent of the power source to the receiver unit and is demodulated there.

The following is an execution example of the invention using the figures addended.

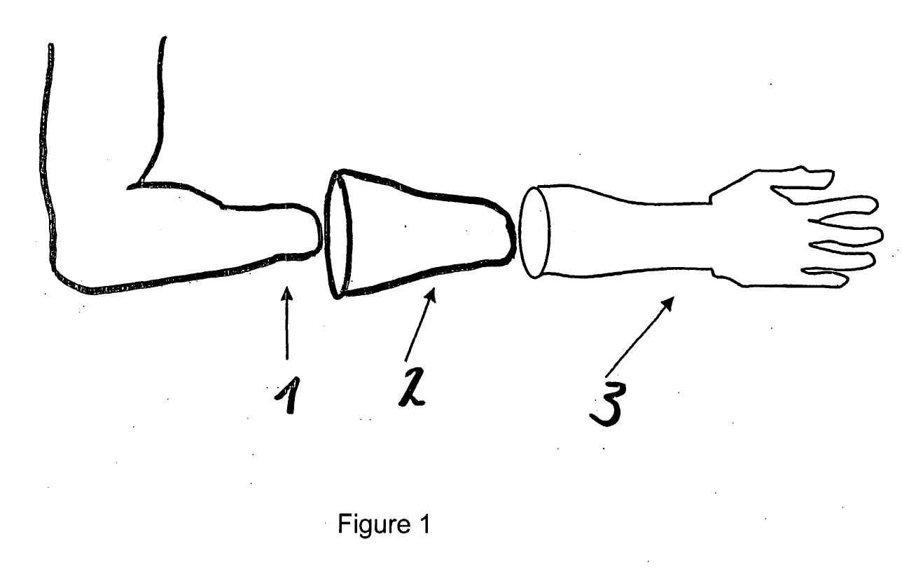

FIG. 1: shows the allocation of the amputation shaft, liner and prosthesis.

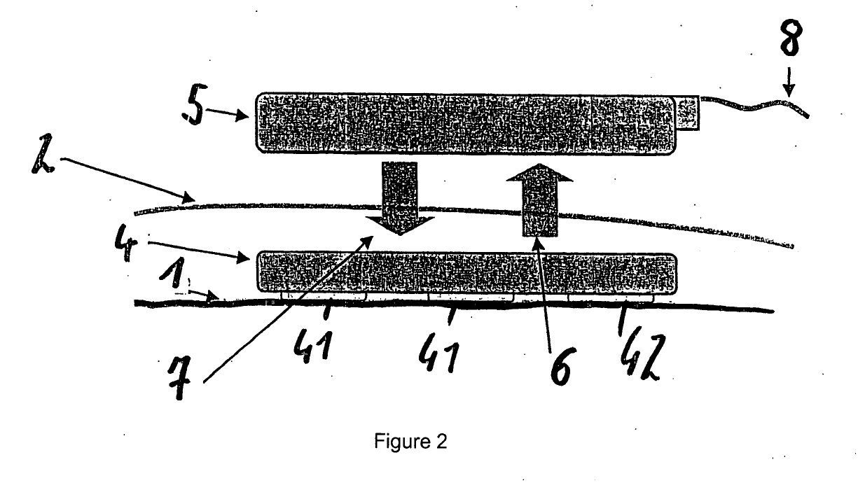

FIG. 2: shows a schematic configuration of the electrode unit on the amputation stump and liner; as well as

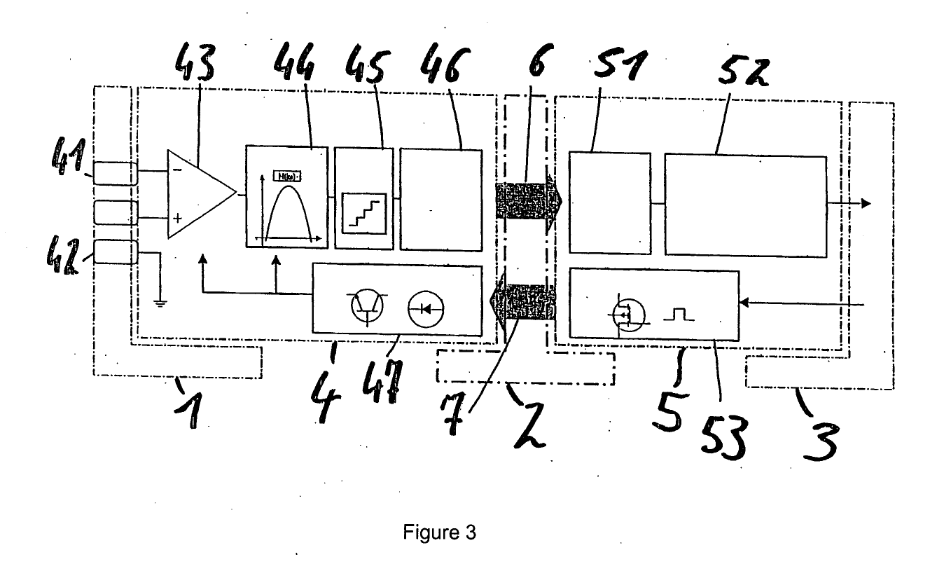

FIG. 3: shows a detailed representation of the functional units of the electrode unit.

FIG. 1 shows the basic configuration of a prosthesis 3 on an amputation stump 1 where there is a liner 3 between the amputation stump 1 and the prosthesis 3. The liner 2, which is made of silicone, polyurethane or other adhesive and protective material, is individually fitted to the amputation stump 1 and put on before attachment of the prosthesis shaft 3. The liner 2 is relatively soft and forms a bond layer between the skin and the amputation stump 1 and the inner cladding of the prosthesis shaft 3.

IN order to enable control of movable components of the prosthesis shaft 3, for example, in the finger area, muscle activity is controlled by an unrepresented myoelectrode signal. The so called surface myogram is absorbed and after processing of the signal these signals serve for the control of the mechatronic components.

FIG. 2 shows a cross section of a schematic configuration of an electrode unit which consists of a measurement unit 4 and a receiver unit 5. The measurement unit 4 is located between the surface of the amputation stump 1 and the liner 2 on the skin surface of the amputation stump 1. Conductor electrodes 41 and a grounding electrode 42 are located immediately on the skin surface of the amputation stump 1 and create myoelectrical signals. These signals are prepared in the measurement unit and conducted wirelessly over a sending unit through the liner 2 to a receiver unit on the other side of the liner. The signal transmission 6 occurs wirelessly via optical signals or alternatively via amplitude modulation of electromagnetic signals.

In order to provide the measurement unit with power, an electromagnetic alternation field 7 is provided to the measurement unit where power is induced via an induction spool.

The signal transmission 6 can also occur via frequency modulated or amplitude modulated load modulation. In this the transmission of the power supply 7 is linked to the transmission 6 of the electromyogram signal. The measurement units 4 detunes a resonance circuit which is used for the transmission 7 of the power supply for the measurement unit 4. The detuning is modulated with the binary signal flow and can be detected and demodulated by the receiver unit 5. Alternatively the signal transmission 6 can also occur via an amplitude modulated load modulation.

In the receiver unit 5 there are units for signal dissemination and receivers which prepare the signals and preferably conduct them via cable 8 to the mechatronic components of the prosthesis 3. The receiver unit 5 is preferable located on the inner side of the prosthesis shaft.

The type and manner of construction of the measurement unit 4 and the receiver unit 5 is shown in FIG. 3. The conductor electrodes 41 and the grounding electrode 42 are located directly on the amputation stump 1. From the conductor electrodes 41 the signal is led via an operation amplifier 43 to a filter 44, from which it is then conducted to a coding, modulating and send unit 46 via an analog digital alternator 45. This coding, modulating and sending unit 46 telemetrically transmits a signal through the liner 2 to a receiver module 51, in which the signal is demodulated and decoded. From the receiver 51 the demodulated, decoded signal is conducted for further signal preparation to a relevant signal processing unit 52 from which it is transmitted via an unrepresented cable to elements within the prosthesis shaft 3.

The receiver unit 5 is attached to the inner side of the prosthesis shaft 3, while the measurement unit 4 is attached to the inner side of the liner 2. Preferable, the measurement unit 4 and the receiver unit 5 are configured in such a way that they are aligned and overlap each other. Through this a good telemetric signal transmission is guaranteed. The signal transmission 6 can occur optically as well as electromagnetically.

In order to power the filter 44, the analog digital converter 45, the coding unit and the sender 46 as well as the operation amplifier 43, a power supply unit 57 is constructed with a rectifier in which the electromagnetic alternating fields sent from the alternator unit are converted. Through this an electromagnetic link is created between the measurement unit 4 and the receiver unit 5 which is used for transmission of the power supply.

1. A myoelectrical electrode system for a prosthetic device comprising:
a liner adapted to receive an amputation stump;
a measurement unit including at least one myoelectrode positioned on an amputation side of the liner to detect myoelectric signals, wherein the measurement unit is adapted to wirelessly transmit detected myoelectrical signals; and
a receiver unit positioned on an opposing side of the liner, which is adapted to receive the transmitted myoelectrical signals from the measurement unit.

2. The system of claim 1, wherein the measurement unit includes an amplifier for amplification of the myoelectrical signal.

3. The system of claim 1 wherein the measurement unit includes an analog-digital converter for digitizing the myoelectrical signal.

4. The system of claim 1 wherein the measurement unit includes a coding unit for coding the myoelectrical signal.

5. The system of claim 1 wherein the measurement unit includes an induction coil and a rectifier to provide power to the measurement unit.

6. The system of claim 1 wherein the measurement unit is attached to the amputation surface of the liner.

7. The system of claim 1 wherein the measurement unit is in a watertight encapsulation.

8. The system of claim 1 wherein the receiver unit is adapted to produce electromagnetic alternating fields and includes an induction coil to transmit energy produced thereby.

9. The system of claim 1 wherein the receiver unit is attached to a prosthesis device.

10. The system of claim 1 wherein the measurement unit includes an optical transmitter and the receiver unit includes an optical receiver.

11. The system of claim 1 wherein the measurement unit and the receiver unit comprise a resonant circuit such that myoelectric signal transmission occurs via a frequency or amplitude modulated load modulation.

12. The system of claim 1 wherein signal transmission occurs through amplitude modulation of a carrier signal created in the transmitter, which is demodulated by the receiver.

13. A prosthetic system comprising:
a continuous liner for receiving an amputee stump;
a prosthetic device for placement over the liner on the amputee stump;
a measurement unit adapted to detect myoelectrical signals from the stump and to transmit the detected myoelectrical signals;
a receiver unit adapted to receive the transmitted myoelectrical signals from the measurement unit and to communicate the received myoelectrical signals to the prosthetic device.

14. A liner for an amputee stump comprising:
an inner surface for receiving the amputees stump
an outer surface for receiving a prosthetic device; and
a myoelectrical measurement unit including at least one myoelectrode disposed on an inner surface of the liner to contact the stump, wherein the measurement unit is adapted to detect and transmit myoelectrical signals from the stump.
